(19) United States
(12) Patent Application Publication
(10) Pub. No.: US 2005/0187592 A1
(43) Pub. Date: Aug. 25, 2005

(54) Combined stimulation for auditory prosthesis

(76) Inventors: Peter Seligman, Essendon (AU); Hugh McDermott, Carlton (AU)

Correspondence Address: JAGTIANI + GUTTAG, 10363-A DEMOCRACY LANE, FAIRFAX, VA, 22030, US

(21) Appl. No.: 11/019,361

(22) Filed: Dec. 23, 2004

(30) Foreign Application Priority Data
Dec. 24, 2003 (AU) 2003907149
Jan. 16, 2004 (AU) 2004900216

(51) Int. Cl. A61N 1/18

(52) U.S. Cl. 607/57

(57) ABSTRACT

A cochlear prosthesis comprises multiple electrodes for stimulating the cochlea. A received sound signal is filtered into frequency channels, and from a subset of the frequency channels pulsatile stimuli are generated to be applied by the electrodes. A modulating signal is also obtained from the received sound signal. High rate stimuli modulated by the modulating signal are generated and applied by at least one of the electrodes.

### CROSS-REFERENCE TO RELATED APPLICATIONS

The present application claims priority from Australian Provisional Patent Application No 2003907149 filed on 24 Dec. 2003 and Australian Provisional Patent Application No 2004900216 filed on 16 Jan. 2004, the contents of which are incorporated herein by reference.

### BACKGROUND

1. Field of the Invention

The present invention relates to cochlear prostheses, and in particular to electrical stimulation of the auditory nerve by an implanted cochlear prosthesis in a manner which produces an improved sound percept for the recipient of the prosthesis.

2. Related Art

Cochlear implants have been developed to assist people who are profoundly deaf or severely hearing impaired, by enabling them to experience a hearing sensation representative of the natural hearing sensation. For most such individuals the hair cells in the cochlea, which normally function to transduce acoustic signals into nerve impulses which are interpreted by the brain as sound, are absent or have been destroyed. The cochlear implant therefore bypasses the hair cells to directly deliver electrical stimulation to the auditory nerve with this electrical stimulation being representative of the sound.

Cochlear implants have traditionally consisted of two parts, an external speech processor unit and an implanted receiver/stimulator unit. The external speech processor unit has been worn on the body of the user and its main purpose has been to detect the external sound using a microphone and convert the detected sound into a coded signal through an appropriate speech processing strategy.

This coded signal is then sent to the receiver/stimulator unit which is implanted in the mastoid bone of the user, via a transcutaneous link. The receiver/stimulator unit processes the coded signal into a series of stimulation sequences which are then applied directly to the auditory nerve via a series or an array of electrodes positioned within the cochlea, proximal to the modiolus of the cochlea. One such cochlear implant is set out in U.S. Pat. No. 4,532,930, the contents of which are incorporated herein by reference.

With improvements in technology it is possible that the external speech processor and implanted stimulator unit may be combined to produce a totally implantable cochlear implant unit that is capable of operating, at least for a period of time, without the need for any external device. In such a device, a microphone would be implanted within the body of the user, for example in the ear canal or within the stimulator unit, and sounds would be detected and directly processed by a speech processor within the stimulator unit, with the subsequent stimulation signals delivered without the need for any transcutaneous transmission of signals. Such a device would, however, still have the capability to communicate with an external device when necessary, particularly for program upgrades and/or implant interrogation, and if the operating parameters of the device required alteration.

Much effort has been dedicated to developing suitable stimulations to be applied by such cochlear implants. Currently employed stimulation techniques, such as the SPEAK, CIS and ACE™ strategies employed by Cochlear Ltd, use selected electrodes of the implanted electrode array to apply square biphasic pulses.

In the SPEAK strategy as described for example in U.S. Pat. No. 5,597,380, the amplitude of numerous frequency bands in the audible range (for example 16 or 20 frequency bands) are determined. A subset of electrodes of the electrode array is tonotopically selected to apply biphasic pulses to selected parts of the cochlea, based on those frequency bands which have the largest amplitude. For example, every 4 ms the six frequency bands having the largest amplitude may be chosen, with six corresponding electrodes being used to apply stimuli to the cochlea. The stimuli are typically presented sequentially. Thus, regardless of the particular electrodes applying stimuli within any one period, an overall stimulation rate produced by the prosthesis and applied to the cochlea remains constant. This stimulation rate tends to be a moderate rate resulting in the SPEAK stimulation strategy being power efficient.

The CIS strategy uses high stimulation rates, up to 12 channels, and a fixed subset of electrodes of the electrode array. By using high stimulation rates, CIS produces a percept which conveys more detailed timing information of speech, to assist in conveying the rapid timing cues in speech. The choice of channels, electrodes and stimulation rates is customised to each user by empirical testing, or mapping, after the prosthesis has been fitted.

The ACE™ strategy divides audible sound into as many as 22 frequency bands. Each frequency band is used to produce stimulations by a specific electrode along the electrode array, once again based on the tonotopic position of that electrode. The stimulation rate produced by the ACE™ strategy varies and may produce as many as 14,400 pulses per second.

While the stimulation strategies currently employed provide for device customisation in order to produce the best available percepts for the prosthesis recipient, it is nevertheless acknowledged in the cochlear implant field that the percepts produced by pulsatile electrical stimulation are often un-natural sounding and somewhat harsh. Although many patients adapt to this sound and, after some time, even find it natural, this is not always the case and some patients may experience difficulties. In any event it would be desirable to apply a stimulation strategy which produces percepts which sound more natural so that patients are not required to go through a stage of adapting to unnatural sounding percepts.

In some instances, patients have reported that analogue stimulation has a more natural sound. Analogue stimulation however has some disadvantages due primarily to channel interaction effects. For instance, when a number of current sources are used simultaneously the electric fields can sum without control beyond a comfort threshold and produce an excessively loud percept.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

According to a first aspect, the present invention provides a cochlear prosthesis comprising:
a plurality of electrodes for stimulating the cochlea;
means to filter a received sound signal into a plurality of frequency channels;
means to generate from at least a subset of said channels pulsatile stimuli to be applied by said plurality of electrodes;
means to obtain a modulating signal from the received sound signal; and
means to generate stimuli modulated by the modulating signal, to be applied by at least one of the plurality of electrodes.

According to a second aspect, the present invention provides a method of generating stimuli for a cochlea comprising:
filtering a received sound signal into a plurality of frequency channels;
generating from at least a subset of said channels pulsatile stimuli to be applied by a plurality of electrodes;
obtaining a modulating signal from the received sound signal; and
generating stimuli modulated by the modulating signal, to be applied by at least one electrode.

According to a third aspect the present invention provides a speech processor for a cochlear prosthesis, the speech processor comprising:
means to filter a received sound signal into a plurality of frequency channels;
means to generate from at least a subset of said channels commands for pulsatile stimuli to be applied by a plurality of electrodes;
means to obtain a modulating signal from the received sound signal; and
means to generate commands for stimuli modulated by the modulating signal, to be applied by at least one electrode.

The stimuli modulated by the modulating signal are preferably at a high rate and amplitude modulated by the modulating signal. Alternatively, the stimuli may be pulse width modulated by the modulating signal.

Preferably, the at least one of the plurality of electrodes by which the high rate stimuli are to be applied is the most apical electrode. Alternatively, the at least one of the plurality of electrodes by which the high rate stimuli are to be applied may be selected based on empirical fitting of the prosthesis following surgical implantation of the prosthesis. The high rate stimuli may be applied by a bipolar pair of electrodes or use one or more intra cochlear electrodes with an extra-cochlear return path.

Preferably, the pulsatile stimuli and the high rate stimuli are applied sequentially, such that only one stimulus is applied by the electrode array at one time. For instance, every second stimulus applied by the electrode array may be a stimulus of the high rate stimuli applied by the at least one of the plurality of electrodes, wherein the pulsatile stimuli make up the remainder of the stimuli applied by the electrode array. Alternatively, every third, fourth or other integer stimulus applied by the electrode array may be a stimulus of the high rate stimuli applied by the at least one of the plurality of electrodes, wherein the pulsatile stimuli make up the remainder of the stimuli applied by the electrode array.

The pulsatile stimuli generated from at least a subset of the channels may be applied in a tonotopic manner such that an electrode to apply each pulsatile stimulus is selected based on tonotopic position of that electrode within the cochlea.

The plurality of frequency channels may comprise 20 frequency channels. The plurality of frequency channels is preferably at substantially even logarithmic spacings throughout a subset of the audible frequency range, for instance, the centre of each frequency channel may be positioned from a low frequency in the range of 120-300 Hz to a high frequency in the range of 5-10 kHz.

Pulsatile stimuli may be generated from those channels which have a large amplitude within a given analysis period. For instance, pulsatile stimuli may be generated based on the six channels of largest amplitude within the analysis period. The channel amplitudes may be obtained by use of a fast Fourier transform (FFT) algorithm. The analysis period may be 4 ms.

The modulating signal may be obtained directly from the received sound signal without processing, or alternatively the received sound signal may be processed in some manner in order to obtain the modulating signal. The modulating signal may be obtained from the received sound signal by band-pass filtering the received sound signal, to exclude components outside a desired frequency range. The desired range may have a lower frequency limit of 340 Hz and a high frequency limit of 2700 Hz. Additionally or alternatively, in obtaining the modulating signal the received sound signal may be compressed, for example to conform to patient threshold and comfort levels of stimulation.

The high rate stimuli may be applied by a plurality of electrodes. In such embodiments, the selection of which electrodes are to be used as high rate electrodes may be based on achieving an appropriate physical distribution of the high rate stimulation.

The plurality of high rate electrodes may be used to physically distribute a single high rate stimulus sequence. For instance, where two high rate electrodes are used to apply the high rate stimuli, one high rate electrode may apply every second high rate stimulus, while the other high rate electrode applies every other high rate stimulus. Similarly, where three high rate electrodes are used to apply the high rate stimuli, a first high rate electrode may apply every third high rate stimulus, a second high rate electrode may apply each following high rate stimulus, and a third high rate electrode may apply each remaining high rate stimulus.

Alternatively, each of the plurality of high rate electrodes may apply a distinct high rate stimulus sequence. In such embodiments, the system preferably comprises means to obtain a plurality of modulating signals from the sound signal, and means to generate a plurality of high rate stimuli, each modulated by one of said modulating signals, whether by amplitude modulation or pulse width modulation. The plurality of modulating signals may comprise modulating signals obtained from distinct frequency components of the sound signal. For instance, three modulating signals may be obtained, comprising a first modulating signal obtained from a low frequency component of the sound signal, a second modulating signal obtained from a medium frequency component of the sound signal, and a third modulating signal obtained from a high frequency component of the sound signal.

In embodiments where the modulating signals have been obtained from distinct frequency components of the sound signal, high rate electrodes to apply each high rate stimulus modulated by one of said modulating signals are preferably selected tonotopically. For example, where three modulating signals are obtained, the high rate electrodes are preferably selected based on their position such that high rate stimuli modulated by the first modulating signal are applied by an apical electrode, high rate stimuli modulated by the third modulating signal are applied by a basal electrode, and high rate stimuli modulated by the second modulating signal are applied by an electrode between the apical electrode and the basal electrode.

The prosthesis may further comprise input processing means, such as one or more of a pre-amplifier, a low pass filter, a bandpass filter, and a speech processing means.

The cochlear prosthesis may comprise 22 electrodes for stimulating the cochlea.

The prosthesis may further comprise user input means enabling a user to control a stimulation strategy. For example the user input means may enable the user to selectively enable high rate modulated stimuli, such as when the user is in a non-speech environment for instance in the presence of music. The user may for example selectively disable high rate modulated stimuli in speech environments should the user thus gain a better speech percept. The user input means may further enable user control of balance between a high rate modulated stimulation strategy and a pulsatile stimulation strategy. Such control may be continuous so as to enable a smooth transition between the strategies in accordance with user preference.

According to a fourth aspect the present invention provides a computer program for generating stimuli for a cochlea comprising:
code for filtering a received sound signal into a plurality of frequency channels;
code for generating from at least a subset of said channels commands for pulsatile stimuli to be applied by a plurality of electrodes;
code for obtaining a modulating signal from the received sound signal; and
code for generating commands for high rate stimuli modulated by the modulating signal, to be applied by at least one electrode.

According to a fifth aspect the present invention provides a computer readable medium having recorded thereon a computer program in accordance with the fourth aspect.

The stimulus commands may comprise radio frequency (RF) frames to be transmitted to an implanted component of an auditory prosthesis.

According to a sixth aspect the present invention provides a method of generating a patient-specific map for a high rate channel of a combined stimulation scheme, the method comprising:
obtaining a multi channel map comprising threshold and comfort levels for a subset of tonotopic electrodes of an electrode array;
while passing a sound signal through the tonotopic electrodes in accordance with a multi channel stimulation scheme, increasing the high rate channel strength from a sub-threshold level, and determining a level at which the patient first obtains a percept;
while passing a sound signal through the tonotopic electrodes in accordance with the multi channel stimulation scheme, reducing the high rate channel strength towards a sub-threshold level, and determining a level at which the patient ceases to obtain a percept;
while passing a sound signal through the tonotopic electrodes in accordance with a multi channel stimulation scheme, increasing the high rate channel strength and determining a comfortable loudness level; and
simultaneously operating the tonotopic electrodes and the high rate electrode at respective comfort levels, and if such simultaneous operation exceeds a combined comfort level of the patient, reducing the multi channel comfort levels and the high rate comfort level.

According to a seventh aspect the present invention provides a computer program for generating a patient-specific map for a high rate channel of a combined stimulation scheme, the computer program comprising:
code for obtaining a multi channel map comprising threshold and comfort levels for a subset of tonotopic electrodes of an electrode array;
code for increasing the high rate channel strength from a sub-threshold level while passing a sound signal through the tonotopic electrodes in accordance with a multi channel stimulation scheme, and determining a level at which the patient first obtains a percept;
code for reducing the high rate channel strength towards a sub-threshold level while passing a sound signal through the tonotopic electrodes in accordance with the multi channel stimulation scheme, and determining a level at which the patient ceases to obtain a percept;
code for increasing the high rate channel strength and determining a comfortable loudness level, while passing a sound signal through the tonotopic electrodes in accordance with a multi channel stimulation scheme; and
code for simultaneously operating the tonotopic electrodes and the high rate electrode at respective comfort levels, and if such simultaneous operation exceeds a combined comfort level of the patient, reducing the multi channel comfort levels and the high rate comfort level.

### BRIEF DESCRIPTION OF THE DRAWINGS

By way of example only, preferred embodiments of the invention will be described with reference to the accompanying drawings, in which:

FIG. 1 is a pictorial representation of a cochlear implant system;

FIG. 2 is a block diagram of a system for combined pulsatile and high rate stimulation in accordance with an embodiment of the invention;

FIG. 3 illustrates combined pulsatile and high rate stimulation in accordance with the system of FIG. 2;

FIG. 4 illustrates high rate stimulation distributed across a plurality of electrodes in accordance with another embodiment of the invention; and

FIG. 5 illustrates modulation of a mapped signal in accordance with a further embodiment of the invention.

### DETAILED DESCRIPTION

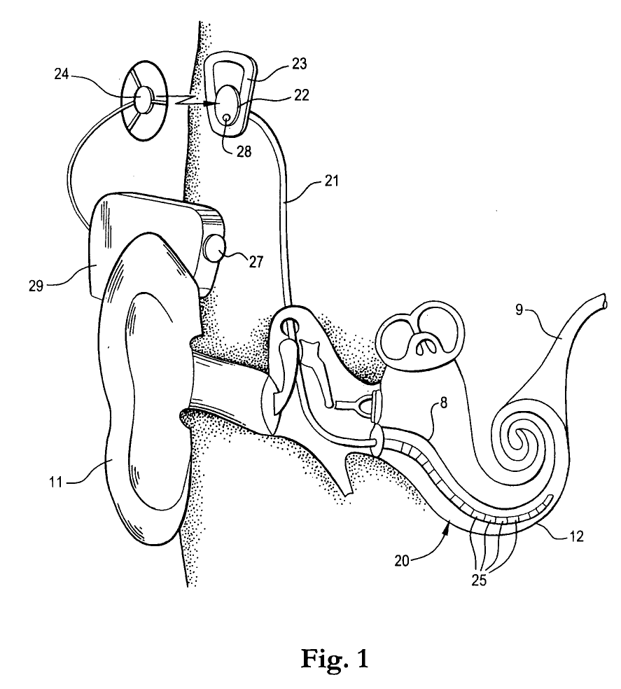

Before describing the features of the present invention, it is appropriate to briefly describe the construction of a cochlear implant system with reference to FIG. 1.

Cochlear implants typically consist of two main components, an external component including a sound processor 29, and an internal component including an implanted receiver and stimulator unit 22. The external component includes an on-board microphone 27. The sound processor 29 is, in this illustration, constructed and arranged so that it can fit behind the outer ear 11. Alternative versions may be worn on the body or it may be possible to provide a fully implantable system which incorporates the speech processor and/or microphone into the implanted stimulator unit. Attached to the sound processor 29 is a transmitter coil 24 which transmits electrical signals to the implanted unit 22 via an RF link.

The implanted component includes a receiver coil 23 for receiving power and data from the transmitter coil 24. A cable 21 extends from the implanted receiver and stimulator unit 22 to the cochlea 12 and terminates in an electrode array 20. The signals thus received are applied by the array 20 to the basilar membrane 8 thereby stimulating the auditory nerve 9. The operation of such a device is described, for example, in U.S. Pat. No. 4,532,930.

The sound processor 29 of the cochlear implant can perform an audio spectral analysis of the acoustic signals and outputs channel amplitude levels. The sound processor 29 can also sort the outputs in order of magnitude, or flag the spectral maxima as used in the SPEAK strategy developed by Cochlear Ltd.

The present invention relates to the combination of the conventional multi-electrode pulsatile paradigms together with a continuous time modulating component.

In one arrangement, the continuous time modulating component is based on a broadband sound signal that modulates a pulsatile carrier, which is then applied to a subset of electrodes. This subset of electrodes is independent of the remaining electrodes, to which conventional pulsatile paradigms are applied.

In another arrangement, the broadband sound signal modulates one or more of a plurality of channels that are generally configured to operate in accordance with conventional pulsatile paradigms.

In another arrangement, a broadband sound signal modulates both a pulsatile carrier that is applied to a subset of electrodes, and one or more of a plurality of channels that are generally configured to operate in accordance with conventional pulsatile paradigms, to the remaining electrodes.

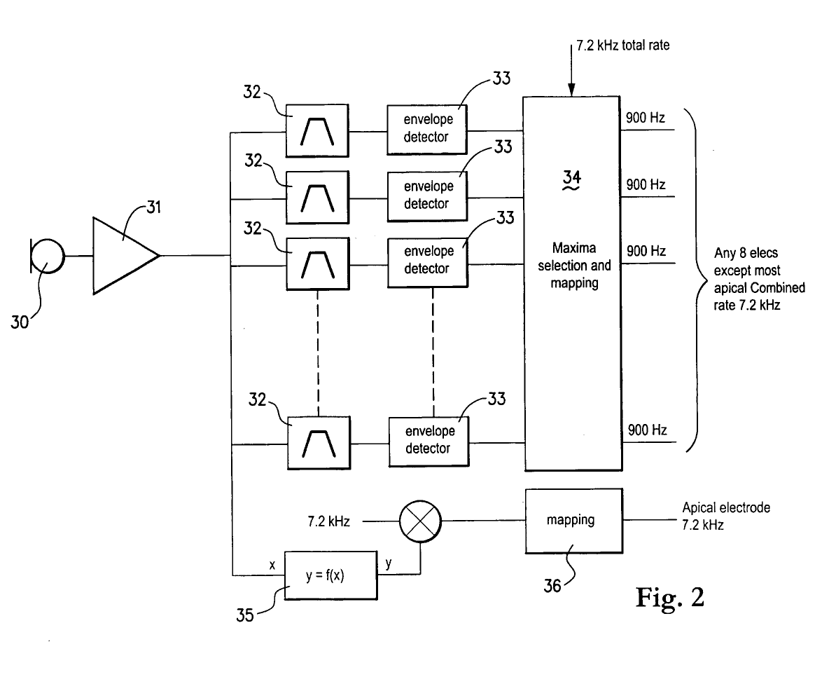

The modulation may be amplitude modulation, pulse width modulation or other form of power modulation conveying the modulating signal. FIG. 2 shows one embodiment of the invention. All channels except the most apical are used in implementing a multi channel strategy, which in the present instance is the SPEAK strategy of Cochlear Ltd. The most apical channel is specially treated as a high rate channel. A microphone 30 detects sound which is passed to preamplifier 31. A plurality of bandpass filters 32 divide the received sound into frequency channels across the audible range of interest. Envelope detectors 33 are used for each channel to assist maxima selection and mapping by processor 34. Every 4 ms 900 Hz stimulation signals are generated based on the eight channels of highest amplitude and passed to eight corresponding electrodes selected tonotopically, with the most apical electrode being excluded from selection for such stimulation.

The amplified detected sound is also passed to a mathematical process 35 which obtains a modulating signal y from the received sound signal x. The modulating signal y is used to amplitude modulate a high rate 7.2 kHz pulse sequence which is mapped by processor 36 and is to be applied by a high rate electrode, in this case being the most apical electrode of the electrode array.

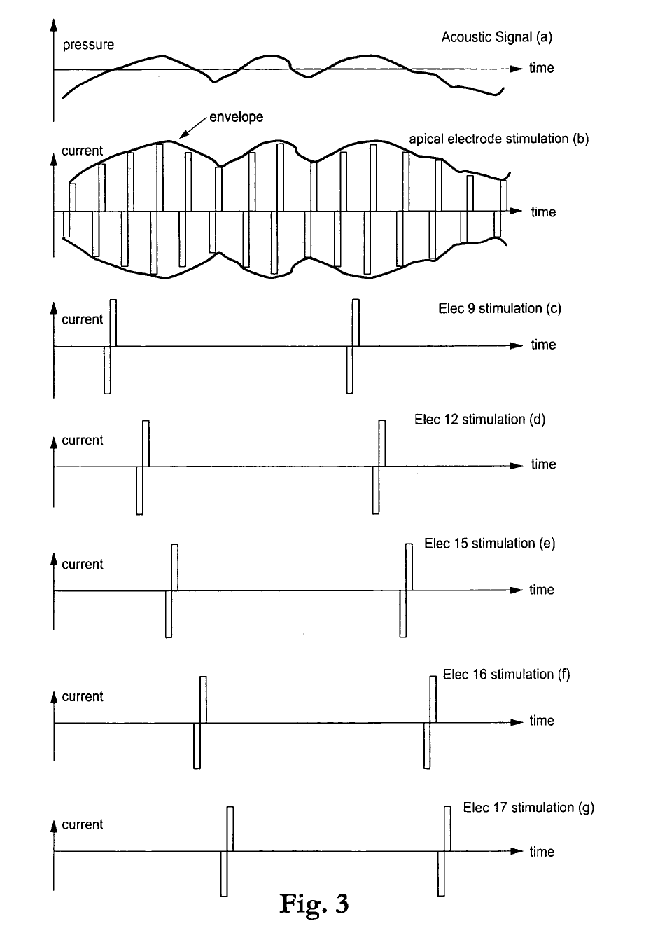

FIG. 3 shows the output of the combined stimulation scheme implemented by the speech processor of FIG. 2. For clarity, only five channels are shown. Trace (a) shows the raw acoustic signal, which may be largely unprocessed, or processed as may be desirable (e.g. band-passed between 340 and 2700 Hz, or compressed). This signal is used to amplitude-modulate a high rate carrier, as shown in Trace (b). Traces (c)-(g) then stimulate in different parts of the cochlea in accordance with a place or tonotopic principle, ie sounds with high frequencies stimulate in the basal region of the cochlea whilst those with low frequency stimulate in the apical region. It is to be noted that stimulations on Traces (c)-(g) convey sound information based on the selection of electrode position. The stimulation rate of the high rate electrode (or “AM channel” electrode) is a multiple of the stimulation rate provided on the electrodes distributed along the cochlea. Each of these channels would be stimulated once whilst the chosen “AM channel” electrode would be stimulated multiple times. In this example, the apical high rate electrode is stimulated on every second occasion, such that the stimulation rate of the apical electrode is eight times the stimulation rate of the eight other stimulating electrodes.

Several alternate embodiments of the invention are possible. For example it would be possible to distribute the region of AM stimulation to a range of electrodes or cochlear places rather than one specific electrode. Thus for example, two or three chosen electrodes would be stimulated, their combined rate being a high rate as in (a) but distributed across these electrodes. As a further alternate, more than one high rate AM carrier may be introduced, each modulated by a different derivative of the acoustic signal, and each receiving more than one pulse per frame. Alternate electrode configurations may also be employed, such as bipolar, tripolar, or arbitrary combinations of electrodes for each channel. It is further to be understood that the pulses employed on any channel need not be rectangular, but may have other shapes as may be advantageous such as sinusoid cycles.

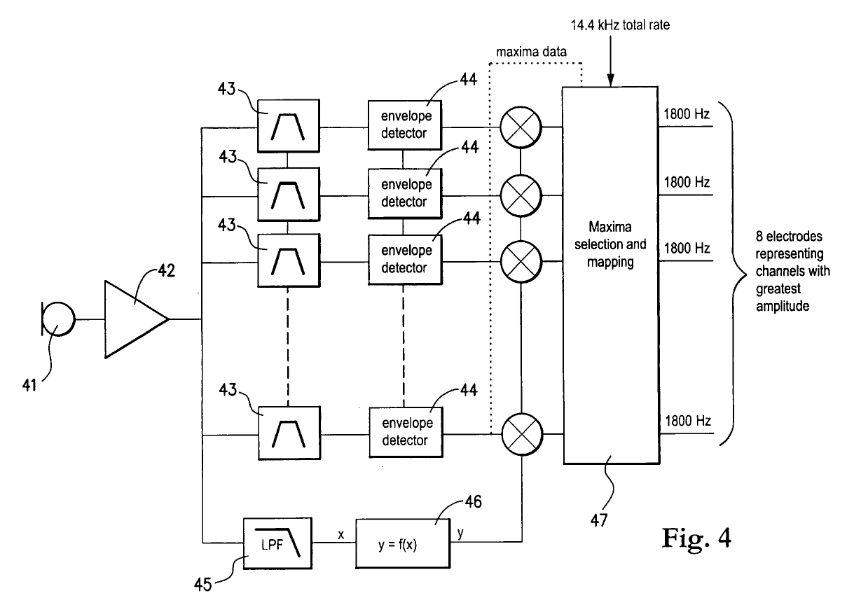

Another embodiment of the present invention is shown in FIG. 4, in which the stimulation rate is distributed over the whole electrode array, as would normally be the case for a multi-channel strategy, but to modulate the smoothed envelopes of the channels with the raw or filtered signal. This scheme is shown in FIG. 4, in which microphone 41 detects sounds and produces a sound signal which is preamplified by preamplifier 42. The amplified received sound signal is then divided into a plurality of frequency channels by bandpass filters 43. Envelope detectors 44 obtain an envelope of each channel, which is then amplitude modulated by a modulating signal y which is obtained by process 46 from a low pass filtered signal x obtained from the input signal by low pass filter 45. Eight chanlels with the highest maxima are then selected by processor 47 for stimulation signals to be sequentially applied by eight tonotopically corresponding electrodes at a stimulation rate of 1800 Hz per electrode. The result is that the entire region stimulated by the electrode array is presented with one unified amplitude modulated envelope. This contrasts with the situation that applies when the outputs of the individual channels are merely smoothed, containing no information of the individual passband frequencies. It contrasts too with the situation where no envelope smoothing is applied because in that case, the phases of the individual filter outputs are not aligned as they are when all channels are modulated by a single signal.

Furthermore, it is assumed that if the multi-channel speech processing algorithm is one which involves the selection of maxima to determine a subset of electrodes to be activated, then this selection will operate on the output of the envelope detectors rather than the modulated output. The sum of all stimulated electrodes would then appear approximately as shown in Trace (b) of FIG. 3. Such a scheme could be applied to all or some of the electrodes.

The input signal may be band-pass or low-pass filtered and then transformed by any mathematical relationship which may be deemed appropriate.

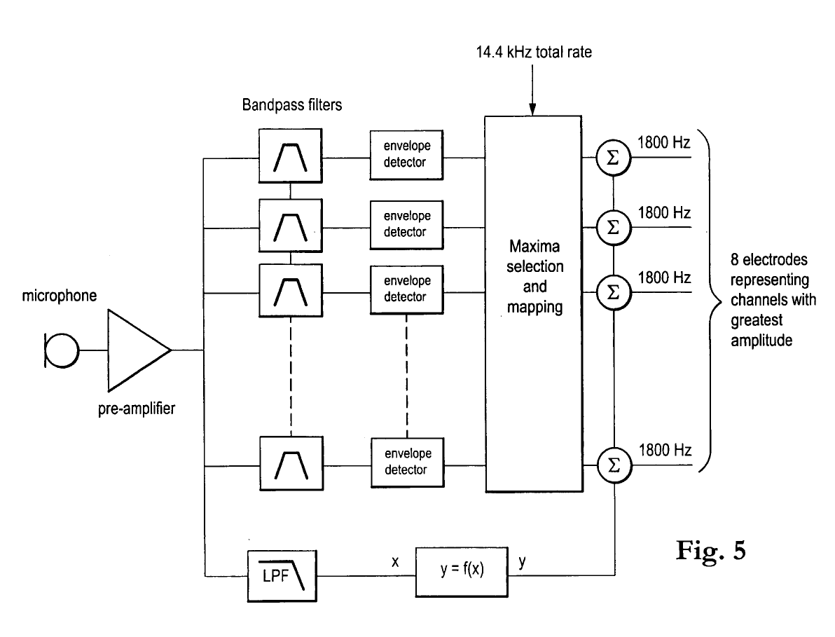

FIG. 5 shows that the input signal may be applied to the mapped signal outputs of the individual channels with or without regard to the individual thresholds and comfortable levels which are applied to each electrode by simply adding or subtracting from them. The input signal would have a variable gain allowing more or less variation of the stimulation levels to be applied according to patient preference or performance measures.

Whilst FIG. 2, FIG. 4 and FIG. 5 show multiplication and addition operators, in each case other operators to combine the signals may be used within the scope of the invention.

Referring to FIG. 2, the method of fitting a patient with the combined single channel and multiple channel map could be as follows:
1. Fit a multiple channel map, as is the normal practice, using all electrodes except the most apical electrode (or other selected single channel).
2. Temporarily, set all thresholds and comfortable levels of the multiple channel map to a sub-threshold level.
3. Run the speech processing strategy, and while it is running, bring up the threshold level of the single channel gradually until the patient can just hear it. Then reduce it until it is just inaudible.
4. With the single channel strategy still running, and some speech input, raise the maximum mapped level, until the speech is heard at a comfortable loudness.
5. Now raise the multiple electrode threshold and comfortable levels to their previously set values so that both strategies are operating simultaneously.
6. If the combined strategy is too loud, lower the levels of each of the individual components as required.

This combined strategy could be used in a number of ways. In one form, the single channel strategy, which for some users may provide more natural sounding percepts, could be chosen for non-speech situations. The patient could switch to a multiple channel strategy for speech communication. At the other extreme, both strategies could be running simultaneously as described above. As a further option, the patient could have the ability to smoothly vary the mix of the two strategies between the single channel and multiple channel cases.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A cochlear prosthesis comprising:
a plurality of electrodes for stimulating the cochlea;
means to filter a received sound signal into a plurality of frequency channels;
means to generate from at least a subset of said channels pulsatile stimuli to be applied by said plurality of electrodes;
means to obtain a modulating signal from the received sound signal; and
means to generate stimuli modulated by the modulating signal, to be applied by at least one of the plurality of electrodes.

2. The cochlear prosthesis of claim 1, wherein the stimuli modulated by the modulating signal are high rate stimuli.

3. The cochlear prosthesis of claim 1, wherein the stimuli modulated by the modulating signal are amplitude modulated by the modulating signal.

4. The cochlear prosthesis of claim 1, wherein the stimuli modulated by the modulating signal are pulse width modulated by the modulating signal.

5. The cochlear prosthesis of claim 1, wherein the at least one of the plurality of electrodes by which the high rate stimuli are to be applied is the most apical electrode.

6. The cochlear prosthesis of claims 1, wherein the at least one of the plurality of electrodes by which the high rate stimuli are to be applied is selected based on post-implantation empirical fitting.

7. The cochlear prosthesis of claims 1, wherein the high rate stimuli are applied by a bipolar pair of electrodes.

8. The cochlear prosthesis of claims 1, wherein the high rate stimuli are applied by one or more intra cochlear electrodes with an extra-cochlear return path.

9. The cochlear prosthesis of claims 1, wherein the pulsatile stimuli and the high rate stimuli are applied sequentially, such that only one stimulus is applied by the plurality of electrodes at one time.

10. The cochlear prosthesis of claim 9, wherein every n^{th} stimulus applied by the electrode array is a high rate stimulus applied by the at least one of the plurality of electrodes, wherein n is an integer greater than one, and wherein the pulsatile stimuli make up the remainder of the stimuli applied by the plurality of electrodes.

11. The cochlear prosthesis of claim 1, wherein the pulsatile stimuli generated from at least a subset of the channels are applied in a tonotopic manner such that an electrode to apply each pulsatile stimulus is selected based on tonotopic position of that electrode within the cochlea.

12. The cochlear prosthesis of claim 1, wherein the plurality of frequency channels comprises 20 frequency channels, at substantially even logarithmic spacings throughout a subset of the audible frequency range.

13. The cochlear prosthesis of claim 1, wherein pulsatile stimuli are generated from those channels which have a large amplitude within a given analysis period.

14. The cochlear prosthesis of claim 13, wherein pulsatile stimuli are generated based on the six channels of largest amplitude within each analysis period.

15. The cochlear prosthesis of claim 1, comprising a band pass filter to obtain the modulating signal from the received sound signal by band-pass filtering the received sound signal, to exclude components outside a desired frequency range.

16. The cochlear prosthesis of claim 15, wherein a lower limit of the desired frequency range comprises substantially 340 Hz.

17. The cochlear prosthesis of claim 15, wherein an upper limit of the desired frequency range comprises substantially 2700 Hz.

18. The cochlear prosthesis of claim 1, comprising a compression means to compress the received sound signal to obtain the modulating signal.

19. The cochlear prosthesis of 1, wherein the high rate stimuli are applied by a second plurality of electrodes of the plurality of electrodes.

20. The cochlear prosthesis of claim 19, wherein a selection of the second plurality of electrodes is made in order to achieve a desired physical distribution of the high rate stimulation.

21. The cochlear prosthesis of claim 19, wherein a single high rate stimulus sequence is physically distributed between the second plurality of electrodes.

22. The cochlear prosthesis of claim 19, wherein each of the second plurality of electrodes applies a distinct high rate stimulus sequence.

23. The cochlear prosthesis of claim 1, comprising input processing means selected from at least one of a pre-amplifier, a low pass filter, a bandpass filter, and a speech processing means.

24. The cochlear prosthesis of 1, comprising user input means enabling a user to control a stimulation strategy.

25. A method of generating stimuli for a cochlea comprising:
filtering a received sound signal into a plurality of frequency channels;
generating from at least a subset of said channels pulsatile stimuli to be applied by a plurality of electrodes;
obtaining a modulating signal from the received sound signal; and
generating stimuli modulated by the modulating signal, to be applied by at least one electrode.

26. The method of claim 25, wherein the stimuli modulated by the modulating signal are high rate stimuli.

27. The method of claim 25, wherein the stimuli modulated by the modulating signal are amplitude modulated by the modulating signal.

28. The method of claim 25, wherein the stimuli modulated by the modulating signal are pulse width modulated by the modulating signal.

29. The method of claim 25, wherein the at least one of the plurality of electrodes by which the high rate stimuli are to be applied is the most apical electrode.

30. The method of claim 25, comprising selecting the at least one of the plurality of electrodes by which the high rate stimuli are to be applied based on post-implantation empirical fitting.

31. The method of claim 25, comprising applying the high rate stimuli by a bipolar pair of electrodes.

32. The method of claim 25, comprising applying the high rate stimuli by one or more intra cochlear electrodes with an extra-cochlear return path.

33. The method of claim 25, comprising applying the pulsatile stimuli and the high rate stimuli sequentially, such that only one stimulus is applied by the plurality of electrodes at one time.

34. The method of claim 33, comprising applying every n^{th} stimulus applied by the electrode array as a high rate stimulus by the at least one of the plurality of electrodes, wherein n is an integer greater than one, and applying pulsatile stimuli to make up the remainder of the stimuli applied by the plurality of electrodes.

35. The method of claim 25, comprising applying the pulsatile stimuli generated from at least a subset of the channels in a tonotopic manner such that an electrode to apply each pulsatile stimulus is selected based on tonotopic position of that electrode within the cochlea.

36. The method of claim 25, wherein the plurality of frequency channels comprises 20 frequency channels, at substantially even logarithmic spacings throughout a subset of the audible frequency range.

37. The method of claim 25, comprising generating pulsatile stimuli from those channels which have a large amplitude within a given analysis period.

38. The method of claim 37, comprising generating pulsatile stimuli based on the six channels of largest amplitude within each analysis period.

39. The method of claim 25, comprising band-pass filtering the received sound signal to obtain the modulating signal from the received sound signal by excluding components outside a desired frequency range.

40. The method of claim 39, wherein a lower limit of the desired frequency range comprises substantially 340 Hz.

41. The method of claim 39, wherein an upper limit of the desired frequency range comprises substantially 2700 Hz.

42. The method of claim 25, comprising compressing the received sound signal to obtain the modulating signal.

43. The method of claim 25, comprising applying the high rate stimuli by a second plurality of electrodes of the plurality of electrodes.

44. The method of claim 43, comprising selecting the second plurality of electrodes in order to achieve a desired physical distribution of the high rate stimulation.

45. The method of claim 43, wherein a single high rate stimulus sequence is physically distributed between the second plurality of electrodes.

46. The method of claim 43, wherein each of the second plurality of electrodes applies a distinct high rate stimulus sequence.

47. The method of claim 25, comprising input processing the received signal, by at least one of pre-amplifying, low pass filtering, bandpass filtering, and speech processing.

48. The method of claim 25, comprising enabling a user to control a stimulation strategy.

49. A speech processor for a cochlear prosthesis, the speech processor comprising:
means to filter a received sound signal into a plurality of frequency channels;
means to generate from at least a subset of said channels commands for pulsatile stimuli to be applied by a plurality of electrodes;
means to obtain a modulating signal from the received sound signal; and
means to generate commands for stimuli modulated by the modulating signal, to be applied by at least one electrode.

50. The speech processor of claim 49, wherein the commands for stimuli modulated by the modulating signal are commands for high rate stimuli.

51. The speech processor of claim 49, wherein the commands for stimuli modulated by the modulating signal are commands for stimuli amplitude modulated by the modulating signal.

52. The speech processor of claim 49, wherein the commands for stimuli modulated by the modulating signal are commands for stimuli pulse width modulated by the modulating signal.

53. The speech processor of claim 49, wherein at least one electrode by which the high rate stimuli are to be applied is a most apical electrode.

54. The speech processor of claims 49, wherein at least one electrode by which the high rate stimuli are to be applied is selected based on post-implantation empirical fitting.

55. The speech processor of claim 49, wherein the high rate stimuli are to be applied by a bipolar pair of electrodes.

56. The speech processor of claims 49, wherein the high rate stimuli are to be applied by one or more intra cochlear electrodes with an extra-cochlear return path.

57. The speech processor of claim 49, wherein the commands for pulsatile stimuli and the commands for high rate stimuli are to be applied sequentially, such that only one stimulus is applied by the plurality of electrodes at one time.

58. The speech processor of claim 57, wherein every n^{th} stimulus to be applied by the electrode array is a high rate stimulus applied by the at least one of the plurality of electrodes, wherein n is an integer greater than one, and wherein the pulsatile stimuli make up the remainder of the stimuli to be applied by the plurality of electrodes.

59. The speech processor of claim 49, wherein the commands for pulsatile stimuli generated from at least a subset of the channels are to be applied in a tonotopic manner such that an electrode to apply each pulsatile stimulus is selected based on tonotopic position of that electrode within the cochlea.

60. The speech processor of claim 49, wherein the plurality of frequency channels comprises 20 frequency channels, at substantially even logarithmic spacings throughout a subset of the audible frequency range.

61. The speech processor of claims 49, wherein the commands for pulsatile stimuli are generated from those channels which have a large amplitude within a given analysis period.

62. The speech processor of claim 61, wherein the commands for pulsatile stimuli are generated only from six channels of largest amplitude within each analysis period.

63. The speech processor of claim 49, comprising a band pass filter to obtain the modulating signal from the received sound signal by band-pass filtering the received sound signal, to exclude components outside a desired frequency range.

64. The speech processor of claim 63, wherein a lower limit of the desired frequency range comprises substantially 340 Hz.

65. The speech processor of claim 63, wherein an upper limit of the desired frequency range comprises substantially 2700 Hz.

66. The speech processor of claims 49, comprising a compression means to compress the received sound signal to obtain the modulating signal.

67. The speech processor of claim 49, wherein the commands for high rate stimuli are to be applied by a second plurality of electrodes of the plurality of electrodes.

68. The speech processor of claim 67, wherein a selection of the second plurality of electrodes is made in order to achieve a desired physical distribution of the high rate stimulation.

69. The speech processor of claim 67, wherein commands for a single high rate stimulus sequence are to be physically distributed between the second plurality of electrodes.

70. The speech processor of claim 67, wherein commands for each of a plurality of high rate stimulus sequences are to be applied by a unique one of the second plurality of electrodes.

71. The speech processor of claim 49, comprising input processing means selected from at least one of a pre-amplifier, a low pass filter, a bandpass filter, and a speech processing means.

72. The speech processor of claims 49, comprising user input means enabling a user to control a stimulation strategy.

73. A computer program for generating stimuli for a cochlea comprising:
code for filtering a received sound signal into a plurality of frequency channels;
code for generating from at least a subset of said channels commands for pulsatile stimuli to be applied by a plurality of electrodes;
code for obtaining a modulating signal from the received sound signal; and
code for generating commands for high rate stimuli modulated by the modulating signal, to be applied by at least one electrode.

74. The computer program of claim 73, wherein the stimuli modulated by the modulating signal are high rate stimuli.

75. The computer program of claim 73, wherein the stimuli modulated by the modulating signal are amplitude modulated by the modulating signal.

76. The computer program of claim 73, wherein the stimuli modulated by the modulating signal are pulse width modulated by the modulating signal.

77. The computer program of claim 73, wherein the at least one of the plurality of electrodes by which the high rate stimuli are to be applied is the most apical electrode.

78. The computer program of claim 73, comprising code for selecting the at least one of the plurality of electrodes by which the high rate stimuli are to be applied based on post-implantation empirical fitting.

79. The computer program of claim 73, comprising code for applying the high rate stimuli by a bipolar pair of electrodes.

80. The computer program of claim 73, comprising code for applying the high rate stimuli by one or more intra cochlear electrodes with an extra-cochlear return path.

81. The computer program of claim 73, comprising code for applying the pulsatile stimuli and the high rate stimuli sequentially, such that only one stimulus is applied by the plurality of electrodes at one time.

82. The computer program of claim 81, comprising code for applying every n^{th} stimulus applied by the electrode array as a high rate stimulus by the at least one of the plurality of electrodes, wherein n is an integer greater than one, and code for applying pulsatile stimuli to make up the remainder of the stimuli applied by the plurality of electrodes.

83. The computer program of claim 73, comprising code for applying the pulsatile stimuli generated from at least a subset of the channels in a tonotopic manner such that an electrode to apply each pulsatile stimulus is selected based on tonotopic position of that electrode within the cochlea.

84. The computer program of claim 73, wherein the plurality of frequency channels comprises 20 frequency channels, at substantially even logarithmic spacings throughout a subset of the audible frequency range.

85. The computer program of claim 73, comprising code for generating pulsatile stimuli from those channels which have a large amplitude within a given analysis period.

86. The computer program of claim 85, comprising code for generating pulsatile stimuli based on the six channels of largest amplitude within each analysis period.

87. The computer program of claim 73, comprising code for band-pass filtering the received sound signal to obtain the modulating signal from the received sound signal by excluding components outside a desired frequency range.

88. The computer program of claim 87, wherein a lower limit of the desired frequency range comprises substantially 340 Hz.

89. The computer program of claim 87, wherein an upper limit of the desired frequency range comprises substantially 2700 Hz.

90. The computer program of claim 73, comprising code for compressing the received sound signal to obtain the modulating signal.

91. The computer program of claim 73, comprising code for applying the high rate stimuli by a second plurality of electrodes of the plurality of electrodes.

92. The computer program of claim 91, comprising code for selecting the second plurality of electrodes in order to achieve a desired physical distribution of the high rate stimulation.

93. The computer program of claim 91, wherein a single high rate stimulus sequence is physically distributed between the second plurality of electrodes.

94. The computer program of claim 91, wherein each of the second plurality of electrodes applies a distinct high rate stimulus sequence.

95. The computer program of claim 73, comprising code for input processing the received signal, by at least one of pre-amplifying, low pass filtering, bandpass filtering, and speech processing.

96. The computer program of claim 73 to comprising code for enabling a user to control a stimulation strategy.

97. A computer readable medium having recorded thereon a computer program in accordance with claim 73.

98. A method of generating a patient-specific map for a high rate channel of a combined stimulation scheme, the method comprising:
obtaining a multi channel map comprising threshold and comfort levels for a subset of tonotopic electrodes of an electrode array;
while passing a sound signal through the tonotopic electrodes in accordance with a multi channel stimulation scheme, increasing the high rate channel strength from a sub-threshold level, and determining a level at which the patient first obtains a percept;
while passing a sound signal through the tonotopic electrodes in accordance with the multi channel stimulation scheme, reducing the high rate channel strength towards a sub-threshold level, and determining a level at which the patient ceases to obtain a percept;
while passing a sound signal through the tonotopic electrodes in accordance with a multi channel stimulation scheme, increasing the high rate channel strength and determining a comfortable loudness level; and
simultaneously operating the tonotopic electrodes and the high rate electrode at respective comfort levels, and if such simultaneous operation exceeds a combined comfort level of the patient, reducing the multi channel comfort levels and the high rate comfort level.

99. A computer program for generating a patient-specific map for a high rate channel of a combined stimulation scheme, the computer program comprising:
code for obtaining a multi channel map comprising threshold and comfort levels for a subset of tonotopic electrodes of an electrode array;
code for increasing the high rate channel strength from a sub-threshold level while passing a sound signal through the tonotopic electrodes in accordance with a multi channel stimulation scheme, and determining a level at which the patient first obtains a percept;
code for reducing the high rate channel strength towards a sub-threshold level while passing a sound signal through the tonotopic electrodes in accordance with the multi channel stimulation scheme, and determining a level at which the patient ceases to obtain a percept;
code for increasing the high rate channel strength and determining a comfortable loudness level, while passing a sound signal through the tonotopic electrodes in accordance with a multi channel stimulation scheme; and
code for simultaneously operating the tonotopic electrodes and the high rate electrode at respective comfort levels, and if such simultaneous operation exceeds a combined comfort level of the patient, reducing the multi channel comfort levels and the high rate comfort level.

100. A cochlear prosthesis substantially as herein before described and with reference to the accompanying drawings.

101. A method of generating stimuli for a cochlea substantially as herein before described and with reference to the accompanying drawings.

102. A speech processor for a cochlear prosthesis substantially as herein before described and with reference to the accompanying drawings.

103. A computer program for generating stimuli for a cochlea substantially as herein before described and with reference to the accompanying drawings.

104. A computer readable medium having recorded thereon a computer program substantially as herein before described and with reference to the accompanying drawings.

105. A method of generating a patient-specific map for a high rate channel of a combined stimulation scheme substantially as herein before described and with reference to the accompanying drawings.

106. A computer program for generating a patient-specific map for a high rate channel of a combined stimulation scheme substantially as herein before described and with reference to the accompanying drawings.
